(19) United States
(12) Patent Application Publication
(10) Pub. No.: US 2008/0243077 A1
(43) Pub. Date: Oct. 2, 2008

(54) Fluid dispenser with uniformly collapsible reservoir

(76) Inventors: Donald B. Bivin, Oakland, CA (US); Joshua W. Kriesel, San Francisco, CA (US); Marshall S. Kriesel, Saint Paul, MN (US); George N. Glavee, Burlingame, CA (US)

Correspondence Address: JAMES E. BRUNTON, ESQ., P. O. BOX 29000, GLENDALE, CA, 91209, US

(21) Appl. No.: 11/732,307

(22) Filed: Apr. 2, 2007

(51) Int. Cl. A61M 37/00

(52) U.S. Cl. 604/131

(57) ABSTRACT

A compact, easy-to-use dispensing device that includes a uniquely configured unitary fluid container formed by a blow-fill-seal process. The container has a collapsible, tapered sidewall of progressively varying wall thickness that, upon being acted upon by an elastic member, will deliver an injectable parenteral fluid contained within the fluid reservoir to the patient at a substantially constant flow rate.

### BACKGROUND OF THE INVENTION

1. Field of the Invention

The present invention relates generally to fluid dispensing devices. More particularly, the invention concerns medicament dispensers for dispensing medicinal fluids to ambulatory patients at a precise rate.

2. Discussion of the Prior Art

A number of different types of medicament dispensers for dispensing medicaments to ambulatory patients have been suggested in the past. Many of the devices seek either to improve or to replace the traditional gravity flow and hypodermic syringe methods which have been the standard for delivery of liquid medicaments for many years.

The prior art gravity flow methods typically involve the use of intravenous administration sets and the familiar flexible solution bag suspended above the patient. Such gravimetric methods are cumbersome, imprecise and require bed confinement of the patient. Periodic monitoring of the apparatus by the nurse or doctor is required to detect malfunctions of the infusion apparatus. Accordingly, the prior art devices are not well suited for use in those instances where the patient must be transported to a remote facility for treatment.

As will be fully appreciated from the discussion that follows, the devices of the present invention are particularly useful in combat situations. The ability to quickly and efficaciously treat wounded soldiers, especially in unpredictable or remote care settings, can significantly improve chances for patient survival and recovery. Accurate intravenous (IV) drug and fluid delivery technologies for controlling pain, preventing infection, and providing a means for IV access for rapid infusions during patient transport are needed to treat almost all serious injuries.

It is imperative that battlefield medics begin administering life saving medications as soon as possible after a casualty occurs. The continuous maintenance of these treatments is vital until higher echelon medical facilities can be reached. A compact, portable and ready-to-use infusion device that could be easily brought into the battlefield would allow medics to begin drug infusions immediately. Additionally, it would free them to attend to other seriously wounded patients who may require more hands-on care in the trauma environment following triage. In most serious trauma situations on the battlefield, IV drug delivery is required to treat fluid resuscitation, as well as both pain and infection. Drug infusion devices currently available can impede the timely administration of IV infusions in remote care settings.

Expensive electronic infusion pumps are not a practical field solution because of their weight, cumbersome size and power requirements. Moreover, today's procedures for starting IV infusions on the battlefield are often dangerous because the attending medic must complete several time consuming steps. The labor intensive nature of current gravity solution bag modalities can prevent medics from attending to other patients also suffering from life threatening injuries. In some cases, patients themselves have been forced to hold infusion bags elevated in order to receive the medication by gravity drip.

With regard to the prior art, one of the most versatile and unique fluid delivery apparatus developed in recent years is that developed by one of the present inventors and described in U.S. Pat. No. 5,205,820. The components of this novel fluid delivery apparatus generally include: a base assembly, an elastomeric membrane serving as a stored energy means, fluid flow channels for filling and delivery, flow control means, a cover, and an ullage which comprises a part of the base assembly.

Another prior art patent issued to one of the present applicants, namely U.S. Pat. No. 5,743,879, discloses an injectable medicament dispenser for use in controllably dispensing fluid medicaments such as insulin, anti-infectives, analgesics, oncolylotics, cardiac drugs, bio-pharmaceuticals, and the like from a pre-filled vial at a uniform rate. The dispenser, which is quite dissimilar in construction and operation from that of the present invention, includes a stored energy source in the form of a compressively deformable, polymeric, elastomeric member that provides the force necessary to controllably discharge the medicament from a pre-filled container which is housed within the body of the device. After having been deformed, the polymeric, elastomeric member will return to its starting configuration in a highly predictable manner.

### SUMMARY OF THE INVENTION

By way of brief summary, one form of the of the present invention for dispensing medicaments to a patient comprises a supporting structure, a semi-rigid, uniquely configured, collapsible unitary container carried by the supporting structure and defining a reservoir having an outlet, a first portion, a second portion and a tapered sidewall interconnecting the first and second portions, the sidewall varying in wall thickness from the first portion to the second portion, a stored energy source operably associated with the unitary container for controllably collapsing the container and an administration set including an administration line interconnected with the outlet port of the reservoir.

With the forgoing in mind, it is an object of the present invention to provide a compact, easy-to-use dispensing device that includes a uniquely configured fluid reservoir having a collapsible sidewall of progressively varying wall thickness that will deliver an injectable parenteral fluid contained within the fluid reservoir to the patient at a substantially constant flow rate.

Another object of the invention is to provide a fluid dispenser of the aforementioned character in which the collapsible sidewall is generally conical in shape.

Another object of the invention is to provide a fluid dispenser of the aforementioned character in which the collapsible sidewall is generally rectangular in shape.

Another object of the invention is to provide a fluid dispenser of the aforementioned character in which the collapsible sidewall is generally oval in shape.

Another object of the invention is to provide a dispenser in which a stored energy source is provided in the form of an elastic body, such as a coil spring that provides the force necessary to continuously and uniformly expel fluid from the uniquely shaped reservoir.

Another object of the invention is to provide a fluid dispenser as described in the preceding paragraphs which embodies a semi-rigid, pre-filled, unitary container that is constructed by a blow-fill-seal process and contains within the sealed reservoir of the container the beneficial agents to be delivered to the patient.

Another object of the invention is to provide a compact fluid dispenser as described in the preceding paragraph for use in controllably dispensing from the container reservoir, fluid medicaments, such as, antibiotics, blood clotting agents, analgesics, and like medicinals at a uniform rate.

Another object of the invention is to provide a fluid dispenser of the class described which is compact and lightweight, is easy for ambulatory patients to use, is fully disposable following its use and is extremely reliable in operation.

Another object of the invention is to provide a fluid dispenser of the character described in the preceding paragraphs in which the collapsible sidewall is tapered.

Another object of the invention is to provide a fluid dispenser of the character described in the preceding paragraphs in which the collapsible sidewall has a selectively varying fold depth.

Another object of the invention is to provide a fluid dispenser of the character described in the preceding paragraph in which the collapsible sidewall has a selectively varying fold pitch.

Another object of the invention is to provide a fluid dispenser of the character described in the preceding paragraphs in which the collapsible sidewall has a selectively varying fold angle.

Another object of the invention is to provide a fluid dispenser of the character described that is of a simple construction that can be used in the field with a minimum amount of training.

Another object of the invention is to provide a fluid dispenser of the class described that will permit infusion therapy to be initiated quickly, at will, at point of care on the battlefield so that the attending medic or medical professional can more efficiently deal with triage situations in austere environments.

Another object of the invention is to provide a fluid dispenser that, due to its pre-filled and self-contained packaging, is inherently less likely to result in an unintentional medication error by the attending pharmacist, nurse or other medical clinician.

Another object of the invention is to provide a fluid dispenser as described in the preceding paragraphs that is easy and inexpensive to manufacture in large quantities.

### BRIEF DESCRIPTION OF THE DRAWINGS

FIG. 1 is a generally perspective, top view of one form of the fluid dispensing device of the present invention for dispensing medicaments to a patient.

FIG. 2 is a generally perspective bottom view of the fluid dispensing device shown in FIG. 1.

FIG. 3 is an enlarged, generally perspective, top view of the fluid dispensing device shown in FIG. 1 as it appears with the top removed and the administration set of the apparatus unfurled.

FIG. 4 is an enlarged, generally perspective, fragmentary top view of the upper portion of the fluid dispensing device shown in FIG. 3.

FIG. 5 is an enlarged, longitudinal, cross-sectional view of the fluid dispensing device shown in FIG. 1.

FIG. 5A an enlarged, generally perspective, exploded view, partly in cross-section of the control portion of the fluid dispensing device shown in FIG. 5.

FIG. 5B an enlarged, cross-sectional view of the selector member of the control portion of the fluid dispensing device shown in FIG. 5A.

FIG. 6 is a longitudinal, cross-sectional view, similar to FIG. 5, but showing the various components of the device as they appear following the fluid delivery step.

FIG. 7 is a top plan view of the collapsible, unitary fluid container component of the fluid dispensing device of the present invention.

FIG. 8 is a cross-sectional view taken along lines 8-8 of FIG. 7.

FIG. 8A is an enlarged view of the area designated in FIG. 8 as “8A”.

FIG. 8B is an enlarged view of the area designated in FIG. 8 as “8B”.

FIG. 8C is an enlarged view of the area designated in FIG. 8 as

FIG. 9 is a top plan view of the cover of the rate control assembly of the fluid dispensing device of the present invention.

FIG. 10 is a cross-sectional view taken along lines 10-10 of FIG. 9.

FIG. 11 is a view taken along lines 11-11 of FIG. 10.

FIG. 12 is a top plan view of the rate control plate of the rate control assembly of the fluid dispensing device of the present invention.

FIG. 13 is a cross-sectional view taken along lines 13-13 of FIG. 12.

FIG. 14 is a view taken along lines 14-14 of FIG. 13.

FIG. 15 is a generally perspective, diagrammatic view illustrating a coil spring in position to act upon a generally cylindrically shaped fluid container having a bellows side wall.

FIG. 16 is a generally perspective, diagrammatic view, similar to FIG. 15, but showing the fluid container having been partially collapsed.

FIG. 17 is a generally perspective, diagrammatic view, similar to FIG. 16, but showing the fluid container having been completely collapsed.

FIG. 18 is a generally perspective, diagrammatic view illustrating a coil spring in position to act upon a generally conically shaped fluid container having a bellows side wall.

FIG. 19 is a generally perspective, diagrammatic view, similar to FIG. 18, but showing the generally conically shaped fluid container having been partially collapsed.

FIG. 20 is a generally perspective, diagrammatic view, similar to FIG. 19, but showing the generally conically shaped fluid container having been completely collapsed.

FIG. 21 is a generally perspective, diagrammatic view illustrating a force acting upon a generally conically shaped fluid container.

FIG. 22 is an enlarged, longitudinal, cross-sectional view of an alternate form of the fluid dispensing device of the invention.

FIG. 23 is a longitudinal, cross-sectional view, similar to FIG. 22, but showing the various components of this latest form of the device as they appear following the fluid delivery step.

FIG. 24 is a cross-sectional view of the collapsible, unitary fluid container component of the fluid dispensing device illustrated in FIGS. 22 and 23 of the drawings.

FIG. 25 is an exploded, cross-sectional view of the upper portion of the collapsible, unitary fluid container component illustrated in FIG. 24.

FIG. 26 is a top view of the upper portion of the collapsible, unitary fluid container component illustrated in FIG. 24.

FIG. 27 is a view taken along lines 27-27 of FIG. 26.

FIG. 28 is a cross-sectional view, similar to FIG. 24, but showing the container in a partially collapsed configuration.

FIG. 29 is a cross-sectional view, similar to FIG. 24, but showing the container in a substantially fully collapsed configuration.

FIG. 30 is a generally perspective view of a differently configured, collapsible side wall of an alternate form of fluid container that can be used in the apparatus of the present invention.

FIG. 31 is a front view of the collapsible side wall illustrated in FIG. 30, the rear view thereof being substantially identical.

FIG. 32 is a side view of the collapsible side wall illustrated in FIG. 30, the opposite side view thereof being substantially identical.

FIG. 33 is a generally perspective view of still a differently configured, collapsible side wall of an alternate form of fluid container that can be used in the apparatus of the present invention.

FIG. 34 is a front view of the collapsible side wall illustrated in FIG. 31, the rear view thereof being substantially identical.

FIG. 35 is a side view of the collapsible side wall illustrated in FIG. 31, the opposite side view thereof being substantially identical.

### DESCRIPTION OF THE INVENTION

Definitions: As used herein, the following terms have the following meanings:

### Unitary Container

A closed container formed from a single component.

### Continuous/Uninterrupted Wall

A wall having no break in uniformity or continuity.

### Elastic Member

An object or device that substantially recovers its original shape when released after being distorted.

### Spring

A collapsible, expandable mechanical device constructed from metal, plastic or composite materials that recovers its original shape after being collapsed or extended

### Biologic

A virus, therapeutic serum, toxin, antitoxin, vaccine, blood, blood component or derivative, allergenic product, or analogous product applicable to the prevention, treatment or cure of diseases or injuries of the human or animal body.

### Hermetically Sealed Container

A container that is designed and intended to be secure against the entry of microorganisms and to maintain the safety and quality of its contents after sealing.

### Drug

As defined by the Food, Drug and Cosmetic Act, drugs are “articles (other than food) intended for the use in the diagnosis, cure, mitigation, treatment, or prevention of disease in man or other animals, or to affect the structure or any function.”

### Drug Product

A finished dosage form (e.g. tablet, capsule, or solution) that contains the active drug ingredient usually combined with inactive ingredients.

### Artificial Blood Substitutes

Blood Substitutes are used to fill fluid volume and/or carry oxygen and other gases in the cardiovascular system. These include volume expanders for inert products, and oxygen therapeutics for oxygen-carrying products.

### Resuscitation Fluids

Infusion of hyperosmotic-hyperoncotic solutions such as hypertonic saline dextran (HSD) as used for resuscitation of traumatic shock and perioperative volume support or as an adjunct to other conventional isotonic crystalloid solutions. Where hypotension is caused by myocardial depression, pathological vasodilatation and extravascation of circulating volume due to widespread capillary leak, a resuscitative effort is attempted to correct the absolute and relative hypovolemia by refilling the vascular tree. Here resuscitation with a small volume of hypertonic-hyperoncotic solution allows systemic and splanchnic hemodynamic and oxygen transport recovery without an increase in pulmonary artery pressure. Alternate types of normotonic, hyperoncotic, hypertonic, and hypertonic-hyperoncotic solutions can be used for systemic hemodynamic recovery.

### KVO

KVO—keeping-the-vein-open—in an IV set up. A phrase that refers to the flow rate of a maintenance IV line established as a prophylactic access.

### Nutritionals

Dietary supplemental enteral nutrition support feeding solutions used for nasoenteric application typically used in nasogastric, nasoduodenal, nasojejunal, or intravenous routes of administration.

### Beneficial Agent

The term ‘beneficial agent’ can include any substance or compound that is biologically active and includes any physiologically or pharmacologically active substance that produces a localized or systemic effect in humans or animals and that can be delivered by the present invention to produce a beneficial and useful result.

### Diluent

A liquid that which dilutes, as in an inert solution used to dilute a medicament. An inert liquid carrier of a beneficial agent.

### Collapsible Container

A dispensing device in which one or more walls of the container are made of a material, which will deform (collapse) when pressure is applied thereto, or a dispensing device having a collapsible or telescoping wall structure.

### Aseptic Processing

The term ‘aseptic processing’ as it is applied in the pharmaceutical industry refers to the assembly of sterilized components and product in a specialized clean environment.

### Sterile Product

A sterile product is one that is free from all living organisms, whether in a vegetative or spore state.

### Blow-Film-Seal Process

The concept of aseptic blow-fill-seal (BFS) is that a container is formed, filled, and sealed as a unitary container in a continuous manner without human intervention in a sterile enclosed area inside a machine. The process is multi-stepped; pharmaceutical grade resin is extruded into a tube which is then formed into a container. A mandrel is inserted into the newly formed container and filled. The container is then sealed, all inside a sterile shrouded chamber. The product is then discharged to a non-sterile area for packaging and distribution.

### Integrally Formed

An article of one-piece construction, or several parts that are rigidly secured together and is smoothly continuous in form and that any such components making up the part have been then rendered inseparable.

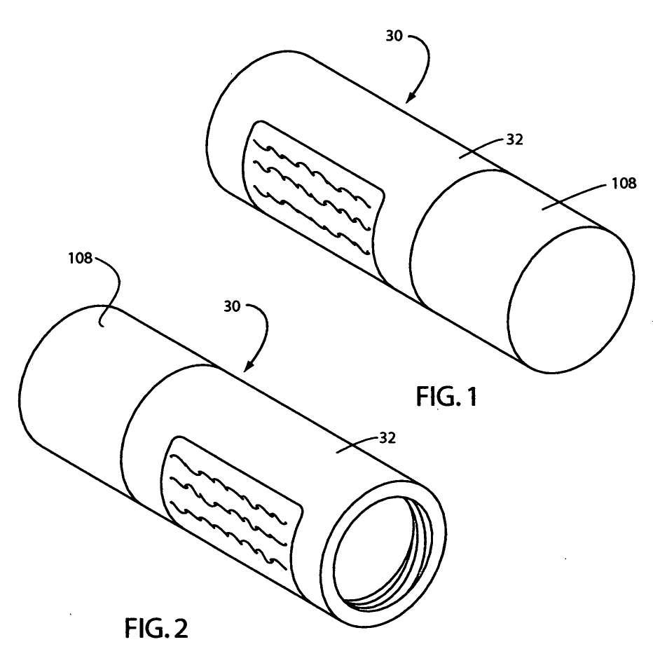

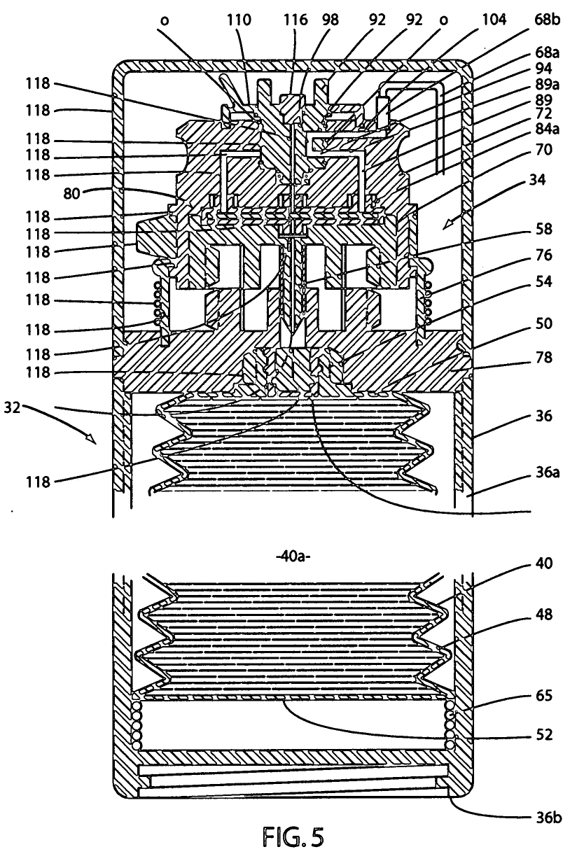

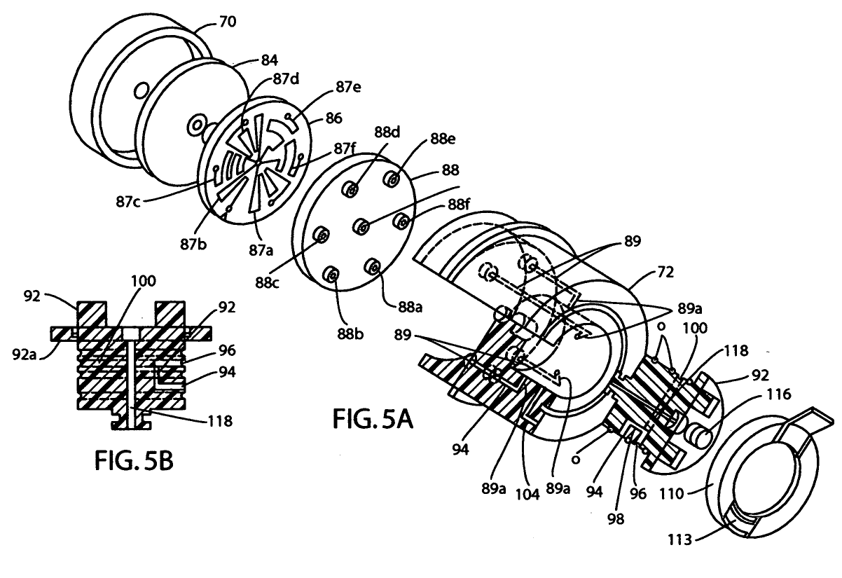

Referring now to the drawings and particularly to FIGS. 1 through 5, one form of the dispensing device of the present invention for dispensing medicaments to a patient is there shown and generally designated by the numeral 30. The dispensing device here includes a housing 32 which includes a control portion 34 and a generally cylindrically shaped reservoir housing 36 that is interconnected with the control portion 34 in the manner best seen in FIG. 5 of the drawings. Housing 32 can be constructed from metal, plastic or any suitable material. Reservoir housing 36 includes a generally cylindrically shaped wall portion 36a and a base portion 36b.

Carried within reservoir housing 36 is a semi-rigid, reservoir-defining assembly, or unitary, pre-filled, hermetically sealed fluid container 40. As best seen by also referring to FIGS. 8, 8A, 8B and 8C, unitary container 40 has an outlet port 42, a first upper portion 44, a spaced-apart second lower, or base, portion 46 and a collapsible, tapered sidewall 48 of progressively varying wall thickness that interconnects first and second portions 44 and 46. In the present form of the invention, the tapered sidewall 48 is continuous and generally accordion-shaped. In a manner presently to be described, side wall 48 is constructed from a yieldably deformable plastic material and uniquely varies in wall thickness from the first portion 44 to the second portion 46. More particularly, as indicated in FIGS. 8A, 8B and 8C, side wall 48 has a first segment 48a of a first wall thickness “x” located proximate base portion 46, a second spaced-apart segment 48b of a second wall thickness “y” greater than said wall thickness “x” and a third segment 48c of a third wall thickness “z” greater than said wall thickness “y”.

As indicated in FIG. 5, in addition to sidewall 48, unitary container 40 includes a top wall 50 and a bottom wall 52. Connected to top wall 50 is a neck portion 54 that is sealed by a closure wall portion 50a.

In the preferred form of the invention unitary container 40 is formed in accordance with an aseptic blow-fill-seal manufacturing technique which is of a character well understood by those skilled in the art. This technique involves the continuous plastic extrusion through an extruder head of a length of parison in the form of a hollow tube between and through two co-acting first or main mold halves. The technique further includes the step of cutting off the parison below the extruder head and above the main mold halves to create an opening which allows a blowing and filling nozzle assembly to be moved downwardly into the opening in the parison for molding the molded container. Further details concerning the technique are available from Rommelag GMBH of Stuttgart, Germany and Weiler Engineering of Elgin, Ill.

As will be described in greater detail hereinafter, the reservoir 40a of the collapsible unitary container 40 is accessible via a penetrating member 58 that is adapted to pierce closure wall 50a as well as a pierceable slit septum 60, which is positioned within neck 54 and over closure wall 50a by means of a closure cap 62, which is affixed to the neck portion 54 of the container assembly by any suitable means such as adhesive bonding or sonic or heat welding.

The fluid contained within the pre-filled unitary container 40 can comprise by way of non-limiting example, a beneficial agent, a drug, a drug substitute, a blood volume expander, a resuscitation fluid, a biologic, blood, an artificial blood substitute, a blood plasma, a nutritional solution, a diluent and a saline solution.

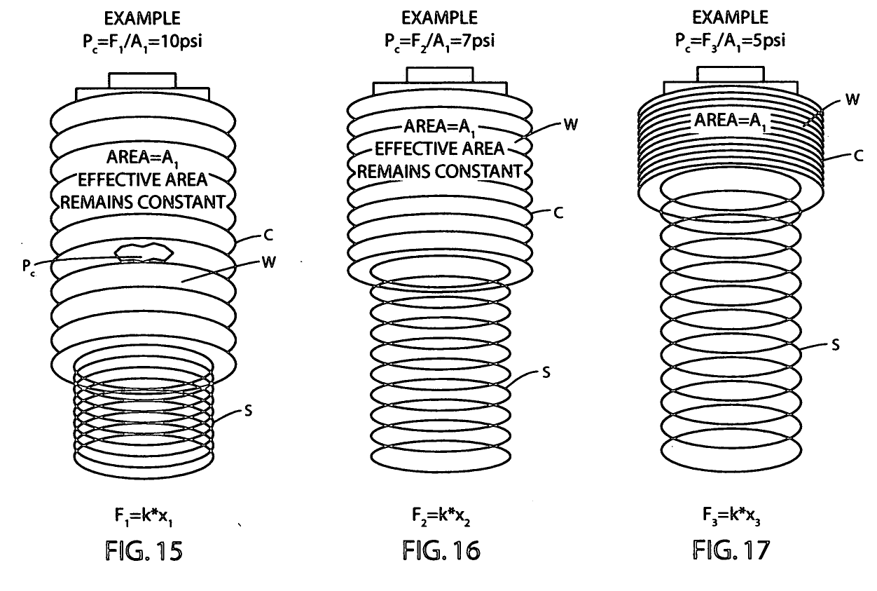

Before discussing further the manner by which the reservoir 40a is accessed, a brief explanation of the importance of the unique shape of the unitary container 40 is in order. Referring to FIGS. 15, 16 and 17 of the drawings, and by way of example, if a standard coil spring, such as spring “S” was used to collapse a generally cylindrically shaped container “C” having a bellows-type side wall “W” defining a fluid containing reservoir “R”, the chamber pressure, that is the pressure within the reservoir “R” of the container, would typically vary according to the properties of the spring. For example, a standard coil spring will generally exhibit a force that displays standard “Hookian” behavior which causes a change in the chamber pressure to vary from 10 psi to 5 psi over the course of the fluid delivery. This phenomenon is undesirable because the change in chamber pressure would result in a proportional change in the fluid flow rate out of the device. As shown in FIGS. 16 and 17, as the force provided by the coil spring “S” changes as it expands to collapse the container “C”, the chamber pressure in the reservoir “R” necessarily would change because the effective area at which the spring engages the reservoir remains the same.

As indicated in FIGS. 16 and 17, the natural characteristics of the spring “S” results in a changing force as it expands so that at any given point during the fluid delivery step, the force per unit area on the container is different. More particularly, at the midway point of the delivery (FIG. 16), the force on the container has changed, but the effective area of the surface touching the spring has not. Accordingly, the chamber pressure will drop, for example, Pc=7 psi (where the effective area acted upon on by the spring is the same).

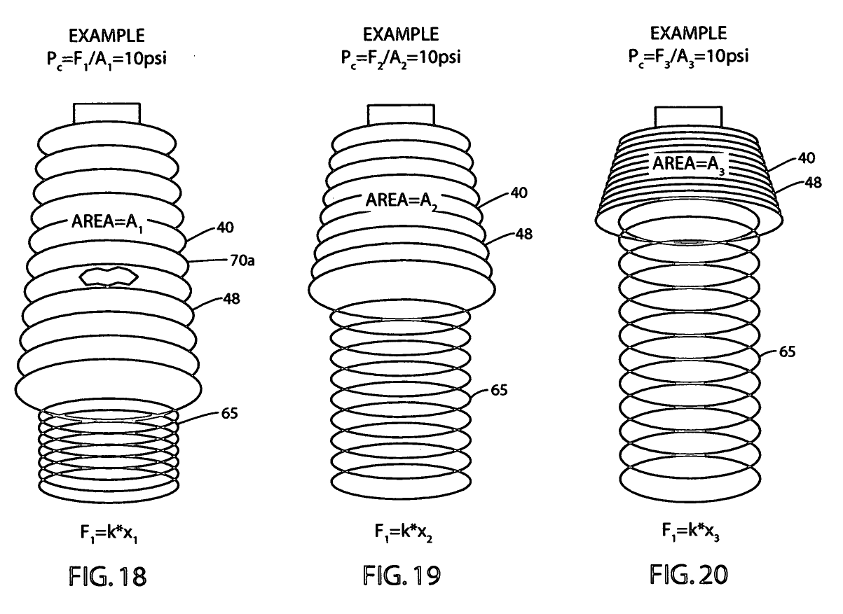

In the apparatus of the present invention, the problem illustrated in FIGS. 15, 16 and 17, is overcome through the use of the uniquely configured unitary container 40 wherein the effective area of the container that the spring 65 is acting upon strategically changes during the course of the spring expansion. More particularly, as illustrated in FIGS. 18, 19 and 20, as the container 40 collapses, the spring force lessens, but so too does the effective area acted upon by the spring. However, because the effective area of the container actually also decreases, by appropriately designing the shape of the container 40 for the particular stress-strain characteristics of the spring 65 being used as the stored energy means, a substantially constant reservoir pressure can be maintained.

As depicted in FIG. 19, the effective area of the unitary container 40 changes as it is collapsed. If this effective area is sized appropriately for the particular characteristics of the spring 65, a constant ratio of force to area (i.e., effective chamber pressure) will result. It is therefore important to note from FIGS. 18, 19 and 20 that:

A1>A2>A3 and F1>F2>F3

Where the effective cross-sectional area of the container that the spring 65 is acting upon is continuously reduced as the container collapses. The unique design of the container and, in particular, the slope of the bellow-shaped sidewall 48 thereof will be configured according to the stress-strain profile of the stored energy source, or spring 65.

As previously discussed, another highly important feature of the present invention resides in the tailoring of the blow-fill-seal process used for making the container 40 to provide a unitary container having a tapered sidewall that exhibits a strategically varying wall thickness. More particularly, the blow-fill-seal process is tailored to provide a unitary container having a tapered sidewall that will be the thinnest at the widest part of the container. This aspect of the blow-fill-seal injection molding process uniquely yields a container that naturally collapses following an applied force starting at the widest portion, that is, the largest area. This will allow the container to exhibit novel collapse dynamics that are appropriately tuned to have an effective area to match the changing magnitude of the spring force.

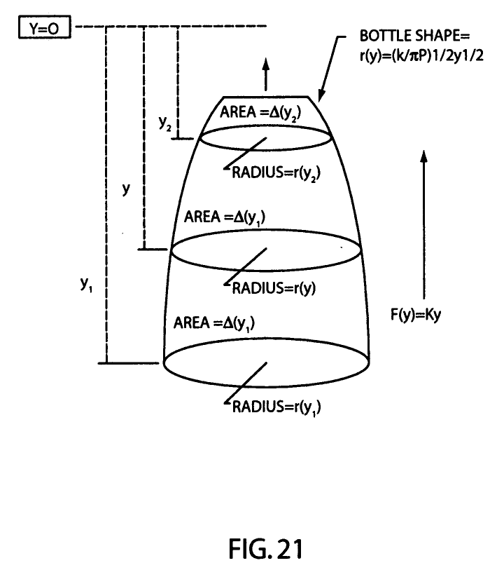

Considering next the relationships between the various parameters required to design a container that will deliver fluid at constant pressure in the case that the force generating the pressure in the container varies linearly as the fluid is delivered from the container. For these considerations, it is assumed that the collapsing force is generated by a simple coiled spring as it extends from a compressed state. It is further assumed, for the purposes of this general example, that the container is circular in cross-section and that the force delivering the fluid decreases by a factor of 2 as the fluid is delivered from the container. Referring to FIG. 21 of the drawings, the variable y lies along the axis of the container and so both the extension of the spring and the compression of the container can be defined by the value of the variable “y”, where y is taken to be the position of the top of the bottle and the position of the moving end of the spring. The variable y decreases in magnitude as the container is compressed and as the spring is extended.

Assume that the relationship between the force and the extension of the spring is given by the expression:

F(y)=ky (1)

Where: the variable y represents the extension of the spring and k is the spring constant. For any value of y the relationship between the pressure, force and area is given by:

P=F(y)/A(y) (2)

Where: F(y) is the force delivered by the spring when the top of the bottle is at position y. A(y) is the cross-sectional area of the bottle at position y. P, the pressure, is independent of y.

The relationship between the cross-sectional area of the bottle and its radius as a function of the variable y is given by:

A(y)=πr(y)² (3)

Using Equations (1), (2) and (3) it we may write:

y=πPr(y)²/k (4)

Rearranging Equation (3) yields an expression for r(y):

r(y)=(k/πP)^{1/2}y^{1/2} (5)

This Equation shows that the radius varies as the square root of the position along the axis.

It can be shown that the volume, V₀, of a container of the present invention (a container with the shape given by Equation (5)) between two values of y (y₁ and y₂) is given

V₀=(k/2P)(y₁²−y₂²) (6)

Where: P is the pressure in the system. Equations (4), (5) and (6) completely specify the container in terms of its shape and length. This can best be illustrated by way of the following two examples:

### EXAMPLE 1

In Example 1, the delivery system design inputs consist of a particular spring (with a specified spring constant), a required container radius and a chamber pressure at which the dispenser will be operated. Therefore, a set of parameters defining the system can be set forth as follows:

The force constant of the spring: k=5 N/cm²

The radius of the container at the position y₁:r₁=2.54 cm

The pressure at which the system will operate: ½ atm=5 N/cm²

With these values Equation (4) yields a value of y₁ as:

y₁=π(Pr₁²)/k=π(5)(2.54)²/5=20.3 cm

If we choose a second value of y, y₂, to be the position where the force is ½ its value at y₁ then we have using Equation 1 that y₂=½ y₁. So that:

y₂=20.15 cm

And the length of the container, L, is then:

L=y₁−y₂=10.15 cm.

Equation (5) gives the shape of the container as:

r(y)=(k/πP)^{1/2}y^{1/2}=(5/π5)^{1/2}y^{1/2}=(1/π)^{1/2}y^{1/2} cm.

Equation (6) gives the volume of the container:

V₀=(½P)(y₁²−y₂²)=( 5/2)(⅕)(20.30²−10.15²)=½(411−103)=154 cm³

Thus, the fluid delivery system would have to have a length of 10.15 cm and a volume of approximately 154 ml—given that the designer wished to use a container with a radius of 2.54 cm, a spring with k=5 N/cm² and a chamber pressure of 0.5 atm.

### EXAMPLE 2

In Example 2, the delivery system design inputs consist of a particular spring (with a specified spring constant), a required container volume and a chamber pressure at which the dispenser will be operated. Therefore, the set of parameters can be set forth as follows:

The volume to be delivered: V₀=250 cm³

The force constant of the spring: k=5 N/cm

The pressure at which the system will operate: P=½ atm=5 N/cm²

For this example we must first solve for y₁ in terms of V₀ We have assumed that: y₂=(½)y₁. So that Equation (6) yields:

V₀(k/2P)(y₁²−y₂²)=(k/2P)(y₁²−(½)²y₁²)=(⅜)(k/P)y₁²

This gives the value of

y₁=( 8/3)^{1/2}(V₀P/k)^{1/2}=( 8/3)^{1/2}(250(5)/5)^{1/2}=((8)( 250/3))^{1/2}=25.82 cm.

And

y₂=(½)(y₁)=25.82/2=12.91 cm

Thus, the length of the container is: L=25.82−12.91=12.91 cm

The shape of the container is given by Equation (4):

r(y)=(k/πP)^{1/2}y^{1/2}=(5/π5)^{1/2}y^{1/2}=(1/π)^{1/2}y^{1/2}

The radius of the container at position #1 can be obtained using Equation (5) and setting y=25.82:

r(y₁)=(1/π)^{1/2}(25.82)^{1/2}=(25.82/π)^{1/2}=2.86 cm

And the radius of the container at position #2 is:

r(y₂)=(1/π)^{1/2}(12.91)^{1/2}=(12.91/π)^{1/2}=2.03 cm

Thus, the container decreases in radius from 2.86 cm to 2.03 cm from the base of the container (contacting the spring) to the tip of the container. In either example, the basic outer shape of the container could also be realized by employing Equation (5).

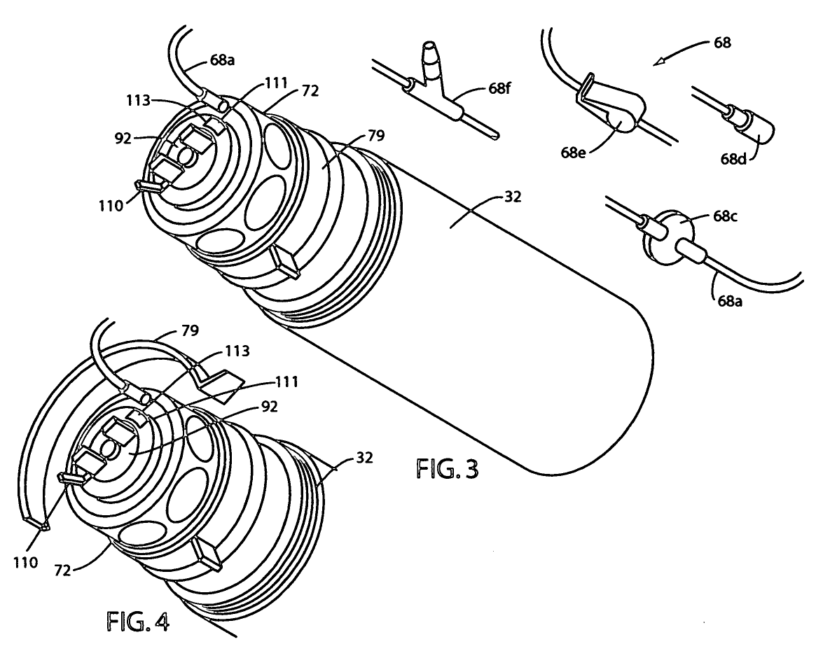

Referring once again to FIG. 5 of the drawings, another important feature of the fluid dispensing device of the invention resides in the provision of flow control means for controlling the flow of medicinal fluid from reservoir 40 of the unitary container toward the administration set 68 of the invention (FIG. 3) and then on to the patient. This novel fluid flow control means here comprises two cooperating components, namely a rate control means for controlling the rate of fluid flow from the collapsible reservoir 40a and a reservoir accessing means for accessing the collapsible reservoir of the device and for controlling fluid flow between the collapsible reservoir and the rate control means.

The reservoir accessing means, which will be discussed in greater detail hereinafter, here comprises a septum-penetrating assembly 70, which includes the previously identified penetrating member 58 (FIG. 5). Septum-penetrating assembly 70 along with selector member housing 72 is movable within a guide sleeve 76 that extends outwardly from a support member 78 that is connected to cylindrically shaped wall portion 36a in the manner shown in FIG. 5. In addition to guiding the travel of the septum-penetrating assembly 70, guide sleeve 76 defines a cylindrical space 76a about which the administration line 68a of the administration set can be coiled in the manner best seen in FIG. 5. Administration set 68 is connected to the selector member housing 72 by a connector 68b in the manner shown in FIG. 5 of the drawings. Disposed between the proximal and distal ends of the administration line is a conventional gas vent and filter 68c. Provided at the distal end is a luer connector 68d of conventional construction (FIG. 3). Between gas vent and filter 68c and luer connector 68d is a conventional line clamp 68e and disposed between gas vent and filter and the proximal end of the administration line is a conventional “Y”-drug infusion site 68f (see FIG. 3).

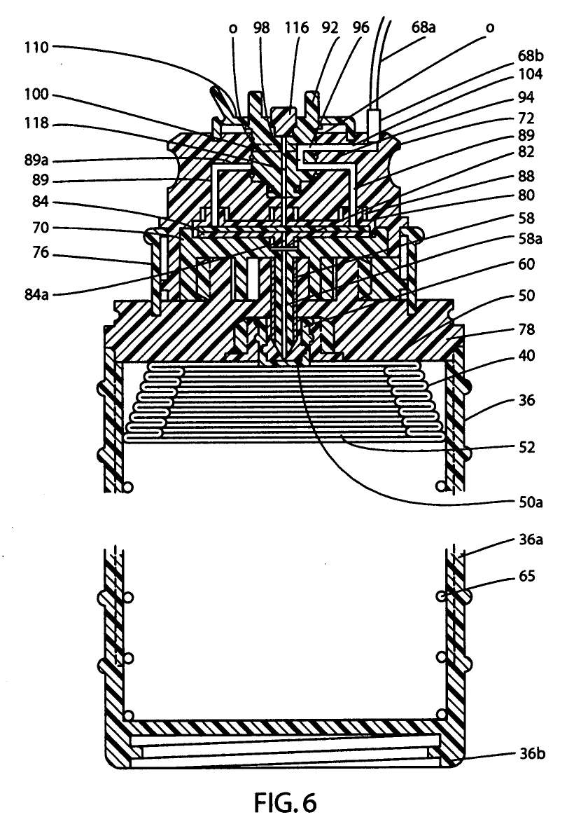

Selector member housing 72 is retained in its first position by a tear strip 79 that is removably receivable between a circumferentially extending rib 72a formed on housing 72 and the upper extremity 76b of guide sleeve 76. When the tear strip 79 is removed in the manner illustrated in FIG. 4, a rotary force exerted on selector member housing 72 will move the housing along with the septum-penetrating assembly into the second extended position shown in FIG. 6 and in so doing will have caused the septum-penetrating member 58 to pierce the septum 60 in the manner shown in FIG. 6. Piercing of the septum 60 and thin wall portion 50a opens a fluid communication path from reservoir 40a to the rate control assembly 80 of the device via a central fluid passageway 58a formed in septum-penetrating member 58. As will be described in greater detail hereinafter, from passageway 58a fluid will flow through conventional particulate filter 82, into inlet 84a of lower rate control cover 84 of the rate control assembly 80, into inlet 86a of rate control plate 86 and then into the various circuitous fluid channels of the rate control plate (see FIG. 14). In a manner to be described in greater detail hereinafter, the fluid will then flow via sealably connected rate control cover 88 into the various circumferentially spaced-apart fluid passageways formed in the selector housing 72 (see FIGS. 5 and 5A).

Considering now in greater detail the rate control assembly 80 of this latest form of the invention, as shown in FIGS. 5A and 14, rate control plate 86 is provided with circuitous fluid channels 87a, 87b, 87c, 87d, 87e and 87f, each of which is of a different geometry including channel length, width and height. As the fluid flows from reservoir 40a into the inlet 86a of rate control plate 86 via rate control cover 84, each of the circuitous fluid channels will fill with the medicinal fluid to be dispensed to the patient. From the circuitous fluid channels, the fluid will flow into outlet passageways 88a, 88b, 88c, 88d, 88e, 88f and 88fg respectively formed in rate control cover 88. From these outlet passageways, the fluid flows into and fills the circumferentially spaced-apart fluid passageways 89 with which they are aligned (see FIG. 5A).

As best seen by referring to FIGS. 5 and 5A and 5B, the selector member 92 of the device is provided with an inlet passageway 94 and an outlet passageway 96 that is interconnected with inlet passageway 94 by means of an axially extending stub passageway 98 which, in turn, is connected to a circumferentially extending channel passageway 100 formed in selector member 92 (FIG. 5B). With this construction, by rotating the selector member, inlet passageway 94 can be selectively brought into index with one of the radial extensions 89a of the axially extending passageways 89 formed in selector member housing 72 thereby providing fluid communication between outlet passageway 96 and the selected one of the circuitous flow passageways formed in rate control plate 86 via annular channel passageway 100 and the selected axially extending passageway 89 formed in the selector member housing 72. Since outlet passageway 96 is in fluid communication with the administration set 68 of the invention via passageway 104 (FIGS. 5, 5A and 6), the rate of fluid flow toward the patient can be precisely controlled by selecting a rate control passageway of appropriate configuration and length, depth and width that is formed in rate control plate 86.

With the device in the configuration shown in FIG. 5, and with the fluid reservoir 40a filled with the medicament to be dispensed to the patient, the dispensing operation can be commenced by removing the top cover 108, which is snapped over support member 78 in the manner shown in FIG. 5. With the cover removed, the administration line 68a of the administration set 68 can be unwrapped from the selector member housing about which it has been coiled. Removal of the top cover also exposes the selector member 92, which is secured in position by a selector member retainer component 110, so that the fluid flow rate can be selected by rotating the selector member to the desired flow rate indicated by the indicia 111 imprinted on the flange 92a of the selector member 92 and is visible through a window 113 provided on the retainer component 110 (FIGS. 3 and 4). Selector member 92 is substantially sealed within the selector member housing 72 by a plurality of O-rings “O”.

In the manner previously described, movement within guide sleeve 76 of the selector member housing 72, along with septum-penetrating assembly 70 from the first position shown in FIG. 5 to the second position shown in FIG. 6 opens fluid communication between reservoir 40a and the rate control assembly 80. This done, the stored energy means, or spring 65, will act upon the unitary container 40 in the manner previously described to collapse the tapered side wall 48 into the collapsed configuration shown in FIG. 6. It is to be understood, that the stored energy means of the present invention for collapsing the unitary container can comprise various types of elastic bodies including springs of various configurations that can be constructed from metal, plastic or composite materials.

To recover any medicament that may remain in reservoir 40a following the fluid delivery step, a pierceable septum 116, which is carried by selector member 92, can be conveniently pierced using a conventional syringe or like device (not shown). Piercing of septum 116 opens communication between reservoir 40a and the syringe via central passageway 118 of the selector member 92, via the rate control assembly 80 and via passageway 58a of penetrating member 58 so that any remaining medicament can be readily recovered from reservoir 40a.

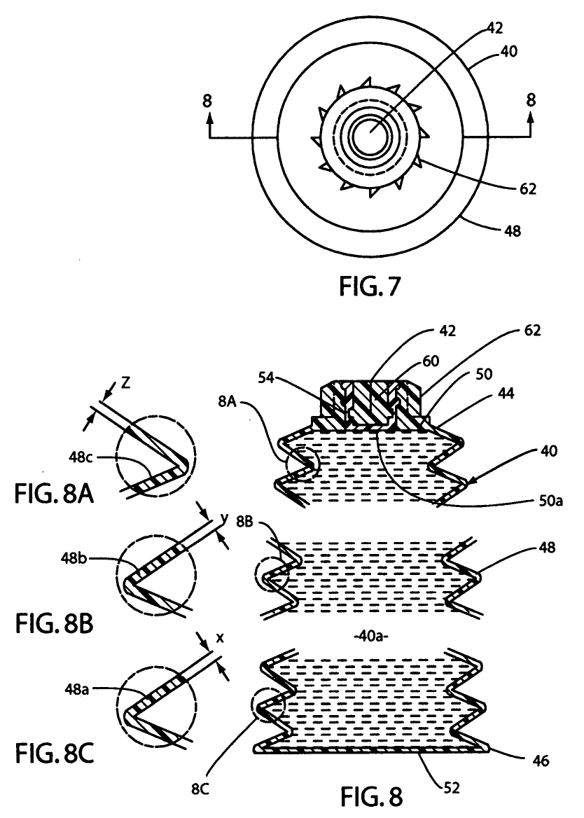

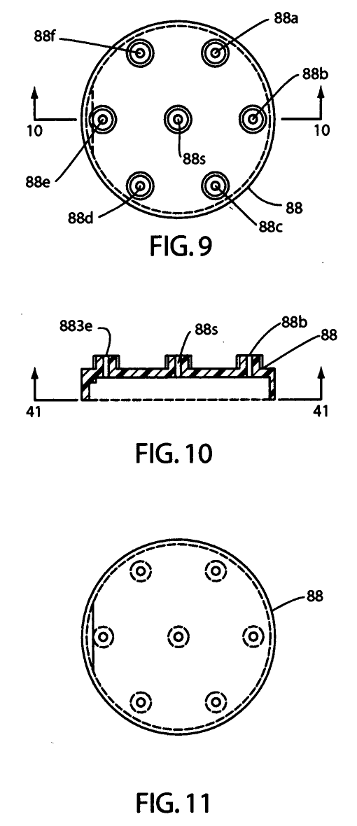

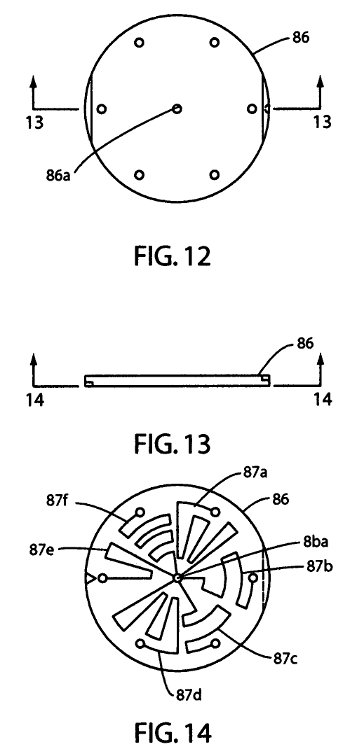

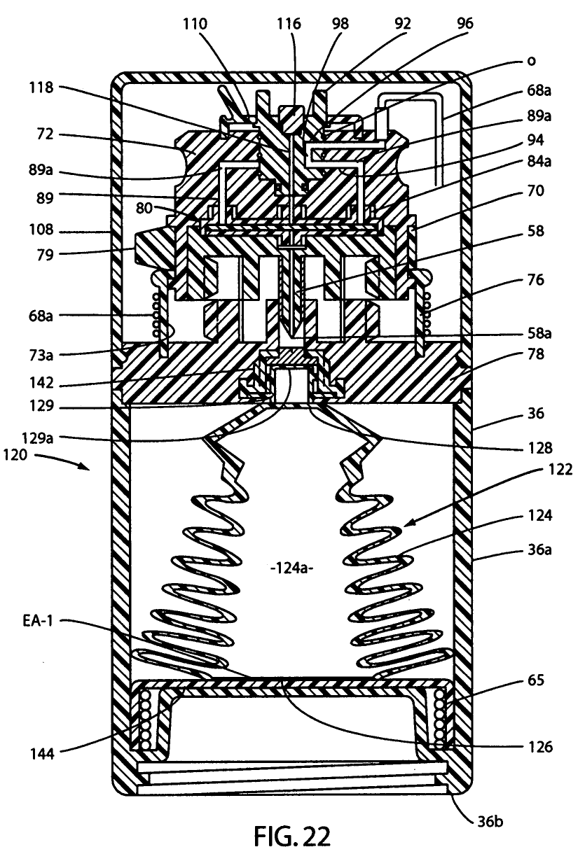

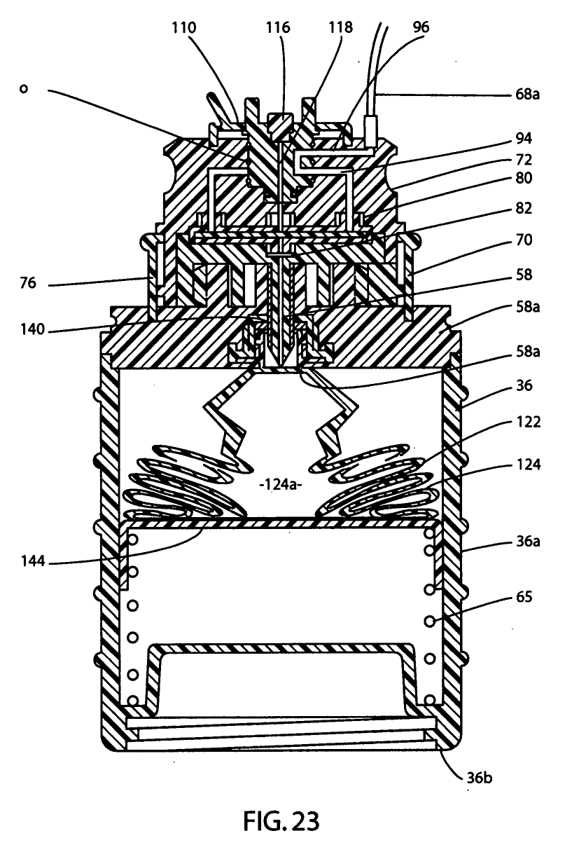

Turning now to FIGS. 22 through 29 of the drawings, an alternate form of the dispensing device of the invention for dispensing medicaments to a patient is there shown and generally designated by the numeral 120. This device is similar and he respects to that shown in FIGS. 1 through 21 of the drawings and light numerals are used in FIGS. 22 through 29 to identify components. As before, this latest form of the dispensing device here includes a housing 32 which includes a control portion 34 and a generally cylindrically shaped reservoir housing 36 that is interconnected with the control portion 34 in the manner best seen in FIG. 23 of the drawings.

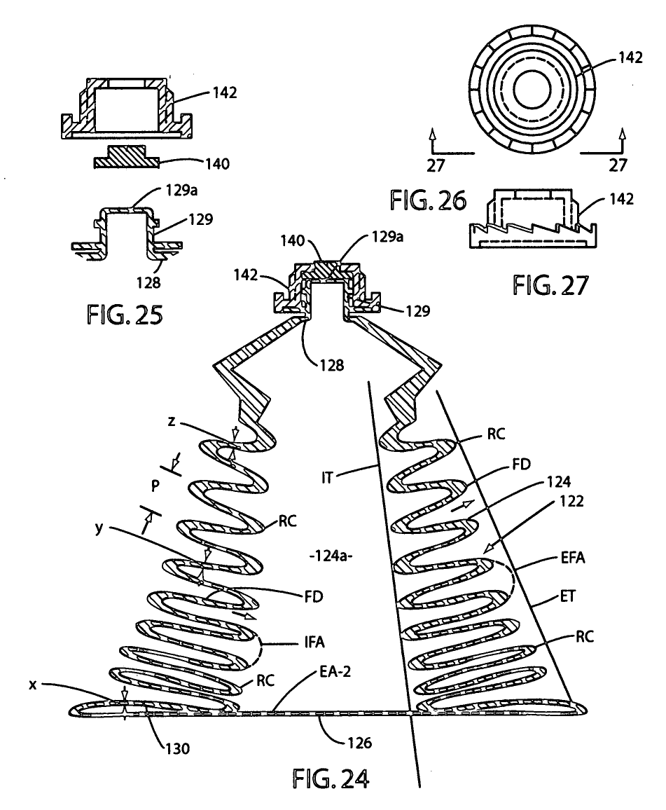

Carried within reservoir housing 36 is a semi-rigid, reservoir-defining assembly, or unitary, pre-filled, hermetically sealed fluid container 122 that is of a somewhat different configuration. More particularly, as will be described in greater detail hereinafter, 122 here comprises a unique tapered, generally bellows shaped, nestable sidewall generally designated by the numeral 124. As illustrated in FIG. 22 the uniquely configured container sidewall extends from the base 126 of the container to the top wall 128 with the thickness of the wall being thinnest proximate the base 126 becoming progressively thicker toward the top wall. More particularly, as depicted in FIG. 24 the wall thickness “x” proximate the base 126 is less than the wall thickness “y” proximate the midpoint of the sidewall. Similarly, the wall thickness “z” proximate the top wall 128 is greater than the wall thickness “y”. Additionally, the fold depth, which is designated as “FD” in FIG. 24 is strategically varied and becomes progressively smaller from the base to the top wall. For present purposes, the fold depth “FD” is defined as the distance between the beginning and the end of any selected fold. As also depicted in FIG. 24, the pitch of the folds, which is generally designated as “p”, becomes progressively larger from the base to the top wall. For present purposes, “pitch” can be defined as the distance between the outermost points of any two adjacent folds. Integrally formed with top wall 128 is a neck portion 129 that is closed by a ceiling wall 129a (see also FIG. 25).

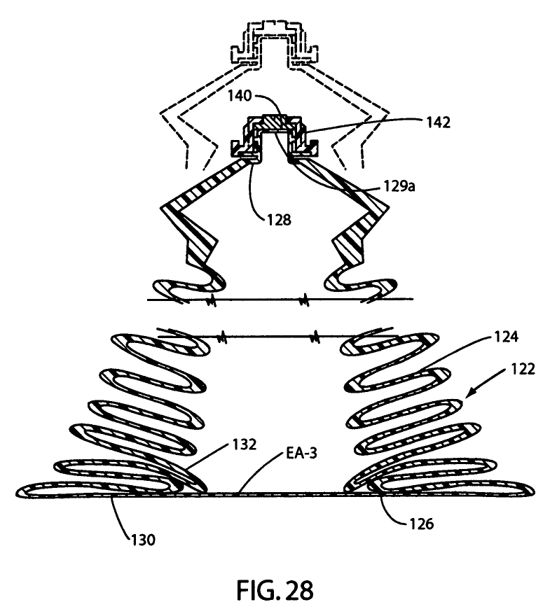

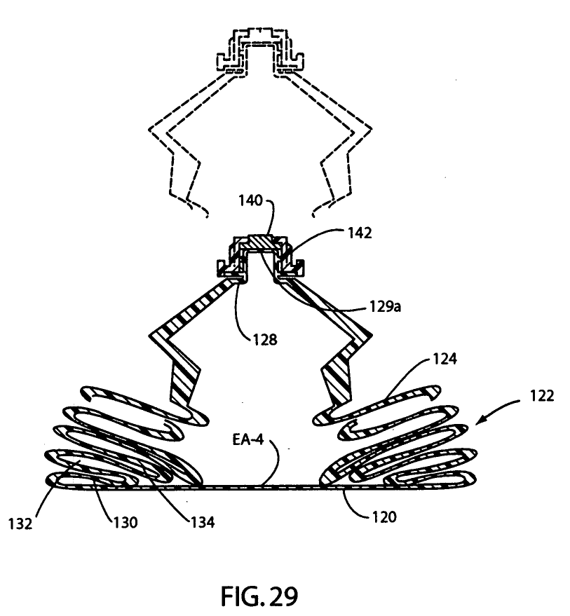

In this latest embodiment of the invention, for the effective area of the unitary container 122 to be reduced as the stored energy source, or spring 65 (FIG. 22) expands and is shown then in more extended position in the manner depicted in FIG. 23 (and provides progressively less force), each successive fold must appropriately “nest” on top of one another so as to “seal-off” a progressively greater area of the container base. That is, each fold must appropriately nest on top of the previously nested fold. This phenomenon, which is illustrated in FIGS. 28 and 29, is what allows the area “A” in the P=F/A (as previously discussed in connection with FIG. 21) to be continuously reduced, thereby maintaining a constant chamber pressure. The foregoing phenomenon is graphically illustrated in FIGS. 24 through 29. More particularly FIG. 24 shows the first fold 130 (i.e. bottom most fold) nesting into or sealing with the base 126. This will reduce the initial effective area “EA-1” of the container (FIG. 22) to “EA-2” (FIG. 24) creating the chamber pressure P=F/A.

Referring to FIG. 28, this figure illustrates the second fold 132 nesting on top of the first fold 130 with this fold, as well as the first fold, being sealed to the base 130. The effective area “EA-3” is now even less than that illustrated in FIG. 24 because in this configuration the two folds 130 and 132 are nested on top of one another—thereby providing a reduced effective (cross-sectional) area.

Turning next to FIG. 29, the third fold 134 is there shown nested on top of the second fold 132 with the third fold 134 also substantially sealed to the base 120 thereby creating even a smaller effective area “EA-4”. It is apparent that, as this folding and nesting progresses with each successive fold collapsing in sequence, the effective area continues to be reduced. It is to be observed that the progressively increasing wall thickness of the tapered sidewall will functions should improve the desired order and priority of fold collapse.

It is also to be observed that the strategic taper angle of the unitary container 122 comprises the central feature that serves to change the effective area of the unitary container-spring interface. More particularly, each successive fold of the container sidewall uniquely nests in a manner previously described, the effective container diameter (and hence the area) increasingly reduces the effective container diameter, the chamber pressure will then be effectively tailored to accommodate the spring dynamics. In this regard, the interior taper, which is designated in FIG. 24 as “IT”, may be defined as the extent to which an imaginary line along the inner folds of the bellows like side wall of the unitary container diverts toward or away from an imaginary line that is perpendicular to the base of the container. Similarly, the exterior taper, which is generally designated in FIG. 24 is “ET” may be defined as the extent to which an imaginary line drawn along the outer folds of the bellows like sidewall of the unitary container diverts toward or away from an imaginary line is perpendicular to the base of the container.

In addition to the thinner walled folds collapsing in priority, the design of a given fold, that is the varying radius of curvature of the fold and the varying fold angle (see FIG. 24), similarly contributes to the desired fold nesting characteristics. In this regard, and shown in FIG. 24, the interior fold angle, which is designated as “IFA”, may be defined as the angle traded by the extremities of two neighboring folds that are on the interior of the container wall. Similarly, the exterior fold angle, which is designated as “EFA” may be defined as the angle created by the extremities of the two neighboring folds that are on the exterior of the container wall. As depicted in FIG. 24, the exterior and exterior fold angles strategically vary along the length of the side wall 124. The varying radius of curvature, which is generally designated in FIG. 24 as “RC”, can be defined as the radius of the circle of curvature of the apex of any selected interior or exterior fold.

With the device in the configuration shown in FIG. 22 of the drawings, and with the fluid reservoir 124a filled with the medicament to be dispensed to the patient, the dispensing operation can be commenced by removing the top cover 108. With the cover removed, the administration line 68a of the administration set 68 can be unwrapped from the selector member housing 76 about which it has been coiled. Removal of the top cover also exposes the selector member 92, which is substantially identical in construction and operation to the previously described in connection with the embodiment of figures and 1 through 14.

In the manner previously described, movement within guide sleeve 76 of the selector member housing 72, along with septum-penetrating assembly 70 from the first position shown in FIG. 22 to the second position shown in FIG. 23. As a septum penetrating assembly moves toward the second position, penetrating member 58 will penetrate the previously identified closure wall 129a thereby opening fluid communication between reservoir 124a and the rate control assembly 80 via a central fluid passageway 58a formed in septum-penetrating member 58. Movement of the septum penetrating assembly toward the second position will also cause penetrating member 58 to penetrate an elastomeric septum 140 that is held in sealing engagement with closure wall 129a by a connector ring 142 that is bonded to, or otherwise affixed to container neck 129 (see FIGS. 24 and 25). This done, the stored energy means, or spring 65, will act upon a carriage 144 that is carried within housing 36 for movement between a first position shown in FIG. 22 and a second position shown in FIG. 23. As carriage 144 moves toward its second position it will act upon container 124 in the manner previously described to collapse, or controllably fold the tapered side wall 124a of the container into the collapsed configuration shown in FIG. 23.

As the sidewall of the unitary container collapses, the fluid contained within reservoir 124a will flow into passageway 58a of the penetrating member 58. From passageway 58a the fluid will flow, and the manner previously described through conventional particulate filter 82, and into the rate control assembly which is substantially of identical construction and operation to that previously described. From the rate control assembly, the medicinal fluid will flow into the various circumferentially spaced-apart fluid passageways formed in the selector housing 72 and then on to the patient via the administration set 68 (see FIGS. 5 and 5A).

As was the case in the earlier described embodiment of the invention, to recover any medicament that may remain in reservoir 124a following the fluid delivery step, a pierceable septum 116, which is carried by selector member 92, can be conveniently pierced using a conventional syringe or like device (not shown).

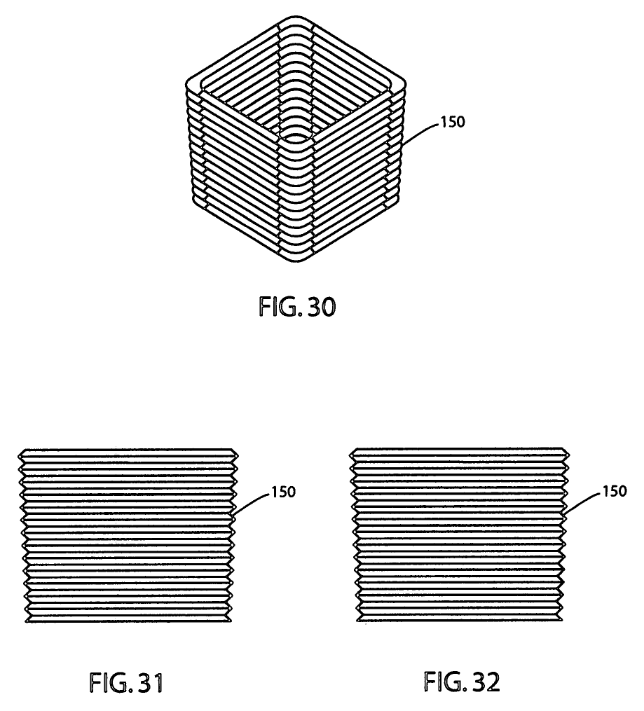

Referring next to FIGS. 30, 31 and 32, there is illustrated a differently configured, generally rectangular-shaped collapsible side wall portion 150 of an alternate form of unitary fluid container of the present invention. As indicated in FIGS. 34 and 35, side wall portion 150 is tapered and, as was the case with the earlier described unitary container 40, is of a progressively varying wall thickness. A unitary fluid container embodying this alternate form of collapsible side wall can be used in lieu of unitary container 40 in an apparatus of the general configuration shown in FIGS. 1 through 5 of the drawings.

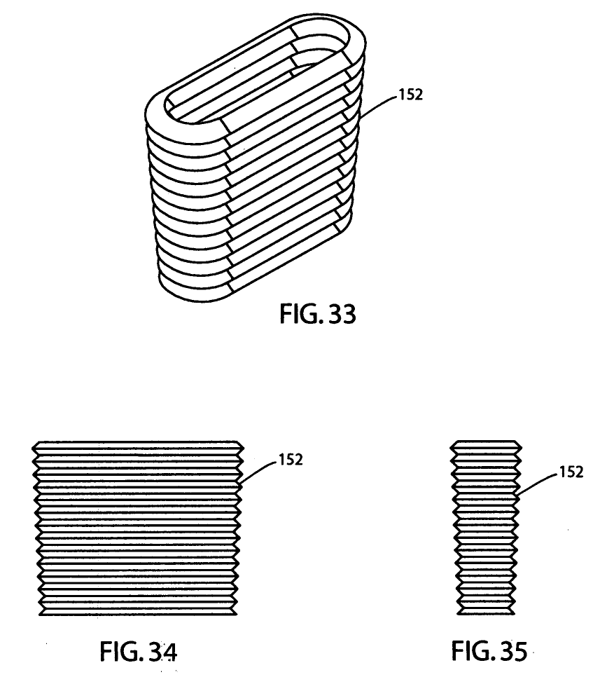

Turning to FIGS. 33, 34 and 35, there is illustrated still another differently configured, generally oval-shaped collapsible side wall portion 152 of yet another alternate form of unitary fluid container of the present invention. As indicated in FIGS. 34 and 35, side wall portion 152 is tapered and, as was the case with the earlier described unitary container 40, is of a progressively varying wall thickness. A unitary fluid container embodying this alternate form of collapsible side wall can also be used in lieu of unitary container 40 in an apparatus of the general configuration shown in FIGS. 1 through 5 of the drawings.

Having now described the invention in detail in accordance with the requirements of the patent statutes, those skilled in this art will have no difficulty in making changes and modifications in the individual parts or their relative assembly in order to meet specific requirements or conditions. Such changes and modifications may be made without departing from the scope and spirit of the invention, as set forth in the following claims.

I claim:

1. A dispensing device for dispensing medicaments to a patient comprising:
(a) a supporting structure;
(b) a pre-filled unitary container formed by an aseptic blow-fill-seal process, said unitary container being carried by said supporting structure and having a tapered collapsible sidewall;
(c) stored energy means carried by said supporting structure and operably associated with said pre-filled unitary container for collapsing said collapsible sidewall thereof; and
(d) flow control means carried by said supporting structure for controlling fluid flow from said unitary container toward the patient.

2. The dispensing device as defined in claim 1 in which said stored energy means comprises a spring operably interconnected with said unitary container.

3. The device as defined in claim 1 in which said collapsible tapered sidewall is of progressively varying wall thickness.

4. The device as defined in claim 1 in which said collapsible tapered sidewall is generally bellows shaped having a plurality of folds of varying pitch.

5. The device as defined in claim 1 said collapsible tapered sidewall is generally bellows shaped having a plurality of folds of varying interior fold angle.

6. The device as defined in claim 1 in which said collapsible tapered sidewall is generally bellows shaped having a plurality of folds of varying fold depth.

7. The device as defined in claim 1 in which said pre-filled unitary container has a base portion and a spaced-apart portion and in which said collapsible sidewall of said unitary container extends between said base portion and said spaced-apart portion and comprises a first segment of a first wall thickness located proximate said base portion and a second spaced-apart segment of a second wall thickness greater than said first wall thickness.

8. The dispensing device as defined in claim 1 in which flow control means comprises rate control means for controlling the rate of fluid flow from said unitary container toward the patient.

9. The dispensing device as defined in claim 8 in which said rate control means includes selector means for selecting the rate of fluid flow from said unitary container toward the patient.

10. The dispensing device as defined in claim 8 in which said flow control means further comprises operating means for controlling fluid flow between said unitary container and said rate control means.

11. The dispensing device as defined in claim 10 in which said sealing means comprises a pierceable member.

12. The dispensing device as defined in claim 11 in which said operating means comprises a penetrating member movable between first position and a second position penetrating said pierceable member thereby permitting fluid flow between said unitary container and said rate control means.

13. A dispensing device for dispensing medicaments to a patient comprising:
(a) a supporting structure;
(b) a pre-filled unitary container carried by said supporting structure and having an outlet port, a first portion, a spaced-apart second portion and a collapsible, tapered sidewall of progressively varying wall thickness interconnecting said first and second portions;
(c) an elastic member operably associated with said unitary container to uniformly collapse said collapsible sidewall;
(d) an administration set, including an administration line interconnected with said outlet port of said unitary container; and
(e) fluid flow control means carried by said supporting structure for controlling fluid flow from said unitary container toward the patient, said fluid flow control means comprising:
(i) rate control means for controlling the rate of fluid flow from said unitary container toward said administration set; and
(ii) operating means for controlling fluid flow between said unitary container and said rate control means.

14. The device as defined in claim 13 in which said unitary container is formed by an aseptic blow-molding process.

15. The device as defined in claim 13 in which said unitary container is formed by an aseptic blow-fill-seal process.

16. The device as defined in claim 13 in which said collapsible sidewall is accordion-shaped.

17. The device as defined in claim 13 in which said collapsible sidewall of said unitary container has a first segment located proximate said first portion of said unitary container and a second, spaced-apart segment, said second spaced-apart segment being of a lesser thickness than said thickness of said first segment.

18. The dispensing device as defined in claim 13 in which said outlet port of said unitary container is sealed by a closure wall.

19. The dispensing device as defined in claim 13 in which said outlet port of said unitary container is sealed by a pierceable septum.

20. The dispensing device as defined in claim 13 in which said operating means comprises a penetrating member movable between first position and a second penetrating position penetrating said closure wall thereby permitting fluid flow between said unitary container and said rate control means.

21. The dispensing device as defined in claim 13 in which said rate control means includes a rate control plate having a plurality of fluid flow channels interconnected with said outlet of said unitary container.

22. The dispensing device as defined in claim 13 in which said elastic member comprises a coil spring.

23. A dispensing device for dispensing medicaments to a patient comprising:
(a) a supporting structure;
(b) a hermetically sealed, unitary collapsible container carried by said supporting structure, said unitary collapsible container being formed using aseptic blow-fill-seal manufacturing techniques and having a pre-filled, sealed fluid reservoir having an outlet port;
(c) a spring operably associated with said collapsible container for collapsing said collapsible container;
(d) an administration set, including an administration line interconnected with said outlet port of said fluid reservoir;
(e) fluid flow control means carried by said supporting structure for controlling fluid flow from said sealed fluid reservoir toward the patient, said fluid flow control means comprising:
(i) rate control means for controlling the rate of fluid flow from said sealed fluid reservoir toward said administration set; and
(ii) operating means for controlling fluid flow between said sealed fluid reservoir and said rate control means.

24. The dispensing device as defined in claim 23 in which said operating means comprises a penetrating member movable between first position and a second position to permit fluid flow between said sealed fluid reservoir of said unitary container and said rate control means collapsible unitary container comprises a bellows structure.

25. The dispensing device as defined in claim 24 in which said bellows structure comprises a plurality of folds of varying wall thickness that are progressively collapsible following movement, of said penetrating member into said second position.

26. The dispensing device as defined in claim 23 in which said pre-filled, sealed fluid reservoir of said collapsible unitary container includes a first portion, a spaced-apart second portion and a collapsible, tapered sidewall of progressively varying wall thickness interconnecting said first and second portions.

27. The dispensing device as defined in claim 26 in which said tapered side wall comprises a first segment of a first wall thickness located proximate said first portion and a second spaced-apart segment of a second wall thickness greater than said first wall thickness.

28. The dispensing device as defined in claim 26 in which the fluid contained within said reservoir comprises a beneficial agent.

29. The dispensing device as defined in claim 26 in which the fluid contained within said reservoir comprises resuscitation fluid.

30. The dispensing device as defined in claim 26 in which the fluid contained within said reservoir comprises a biologic.

31. The dispensing device as defined in claim 26 further including a pierceable septum connected to said collapsible container.
